(19) United States
(12) Patent Application Publication
(10) Pub. No.: US 2006/0216247 A1
(43) Pub. Date: Sep. 28, 2006

(54) Process for treating teeth

(76) Inventor: Eleanor V. Phillips, Rush, NY (US)

Correspondence Address: HISCOCK & BARCLAY, LLP, 2000 HSBC PLAZA, ROCHESTER, NY, 14604-2404, US

(21) Appl. No.: 11/391,926

(22) Filed: Mar. 28, 2006

Related U.S. Application Data
(60) Provisional application No. 60/665,785, filed on Mar. 28, 2005.

(51) Int. Cl. A61K 8/21; A61K 8/34

(52) U.S. Cl. 424/52

(57) ABSTRACT

An acidic solution is applied to the tooth surface in the oral cavity and has the ingredients thymol, eucalyptol and menthol. It kills bacteria in the mouth and makes the environment acidic. The acidity in the oral cavity causes demineralization to occur, releasing calcium and phosphate through open pores in the tooth surface. The acidic solution is removed from the oral cavity after a predetermined amount of time. The non-acidic mouthwash is applied to the tooth surface subsequent to the removal of the acidic solution. It has sodium fluoride and works to neutralize acidic conditions in the oral cavity allowing remineralization to occur. Calcium and phosphate ions fill the open pores in the tooth surface, hardening the enamel. After a predetermined amount of time the user removes the non-acidic solution.

### CROSS-REFERENCE TO OTHER APPLICATIONS

This application claims the benefit of the priority date of U. S. provisional patent application Ser. No. 60/665,785 filed Mar. 28, 2005.

### FIELD OF THE INVENTION

The present invention relates generally to a process of treating teeth. More particularly, the present invention relates to a process of killing bacteria in the oral cavity and demineralizing and remineralizing the tooth surface.

### BACKGROUND OF THE INVENTION

Bacteria are present in the oral cavity and live on the surface of teeth. Young children, who have no teeth, have no bacteria. Bacteria begin to form once the first tooth arrives at about eight months old. Regular bacteria can mutate or generate conditions that support or form harmful anaerobic bacteria. These bacteria accumulate on the tooth surface forming layers known as plaque. The anerobic bacteria live and grow favorably in acidic environments. They also contribute to the acidic environment by metabolizing sugars (from the diet) into additional acid. When this occurs, the pH in the oral cavity drops allowing demineralization to occur.

The tooth is protected by an outer layer of enamel. Enamel is made of 95% mineral surrounded by 5% water. The main mineral, hydroxyapatite, has a crystal structure and is made of calcium and phosphate ions. Acidic conditions in the mouth promote the demineralization of teeth and cause lesions in the enamel to form, eventually leading to cavities. During demineralization, calcium and phosphate ions diffuse out of the tooth surface causing the hydroxyapatite crystals to shrink. Once the acidic conditions neutralize, demineralization stops and remineralization occurs.

Remineralization allows available calcium and phosphate ions to enter lesions that form as a result of demineralization. This process strengthens the teeth and prevents lesions from forming a cavity. Demineralization and remineralization are a dynamic process in the mouth. Cavities will not form as long as the occurrence of demineralization is less than or equal to that of remineralization. A variety of dentifrices, including mouthwash, are used to increase the rate and frequency of remineralization.

Many people use mouthwash to cure bad breath and kill bacteria found in the mouth. It is frequently used in addition to brushing and flossing teeth. There is a large variety of mouthwashes on the market that contain many different ingredients. One popular type of mouthwash is available in a liquid solution at a low pH. Another version is non-acidic and has no alcohol. The mouthwashes have different methods of treating teeth because they contain different ingredients. The acidic mouthwash works to kill bacteria in the oral cavity while the non-acidic mouthwash helps prevent cavities from forming.

Two commonly used brands of mouthwash are FRESHBURST® LISTERINE® and ACT®. LISTERINE® Antiseptic Mouthwash is a registered trademark of Warner-Lambert Consumer Healthcare. It is a popular wash that kills bacteria found in the mouth in addition to curing bad breath. The active ingredients of LISTERINE® include Thymol, Eucalyptol, Menthol and Methyl Salicylate. The solution has a pH of 4.2 and is acidic due to a combination of Thymol, Eucalyptol and Menthol. When a consumer uses an acidic mouthwash to rinse, the pH level in the oral cavity becomes acidic creating an ideal environment for bacteria to grow. Although mouthwash is helpful in killing bacteria it is counter effective when it promotes demineralization and bacterial growth.

LISTERINE® deprives the oral cavity of saliva in addition to creating acidic conditions. LISTERINE® has 21.6% alcohol which acts to dry up saliva. Alcohol is included in various mouthwashes to prevent the product from becoming cloudy. Saliva has calcium, phosphate and fluoride ions that allow the oral cavity to remineralize naturally once the pH level rises above 5.5. Without saliva or with a reduced amount, there may not be enough calcium, phosphate and fluoride for remineralization to occur. Additionally, when saliva is dried up it is unavailable to wash away any food or foreign particles left on the tooth. As such, a mouthwash may be effective at killing bacteria but may do so at the expense of promoting bacterial growth and drying up saliva. To make such mouthwashes more effective, a method of raising the pH in the oral cavity and promoting remineralization is needed.

ACT® is another popular brand of mouthwash. It a registered trademark of Johnson and Johnson and differs from LISTERINE® because it is non-acidic and does not lower the pH in the oral cavity. It also does not kill bacteria. ACT® neutralizes acidic conditions in the mouth and prevents cavities by remineralizing teeth. It has sodium fluoride which enhances the mouth's ability to collect calcium and phosphate ions to fill open pores in the tooth surface.

Fluoride is a very important ingredient in dental treatment and plays a large role in remineralization. Fluoride is present in saliva in low amounts. It promotes remineralization and lowers the solubility of enamel in acid. Fluoride enters the water layer around the minerals in teeth and promotes crystal growth using released calcium and phosphate also found in the layer. A fluoride ion is incorporated into the tooth crystal by replacing a hydroxyl group in the hydroxyapatite structure to form fluoroapatite. Fluoroapatite is less soluble in acid than hydroxyapatite and prevents degradation of the enamel by creating a stronger structure.

ACT® and LISTERINE® are both useful products; however they lack certain aspects that are important in properly caring for teeth. An effective mouthwash kills bacteria to prevent them from metabolizing sugars and creating high acidic conditions in the mouth. ACT® is unable to kill the bacteria and LISTERINE® is effective in doing so but at the expense of lowering the pH in the oral cavity fostering an environment where bacteria may grow. If LISTERINE® is left on the tooth surface the acidity dissolves the tooth crystals leaving the tooth vulnerable to bending and decay. An effective mouthwash also remineralizes teeth. ACT® remineralizes teeth however, it does not kill bacteria. This poses a problem because the bacteria that create the need for remineralization are living and producing acid. Therefore, what is needed is a treatment for teeth that kills bacteria and remineralizes the tooth surface.

### SUMMARY OF THE INVENTION

In the present invention two different mouthwashes are used in a particular sequence to kill bacteria, prevent re-growth and remineralize teeth. The first mouthwash (mouthwash #1) has a low pH and kills bacteria in the mouth. It creates acidic conditions in the oral cavity and causes demineralization to occur. Mouthwash #1 is similar to many of the different types of mouthwash on the market today including LISTERINE®. The user applies mouthwash #1 to the tooth surface by placing a predetermined amount in the mouth and allowing it to flow over the surface of teeth. The user disposes of mouthwash #1 after a predetermined amount of time. The second mouthwash (Mouthwash #2) is non-acidic should be applied to the tooth surface within 30 minutes after disposing of mouthwash #1. The user places a predetermined amount of Mouthwash #2 into the mouth and allows it to flow over the tooth surface. Mouthwash #2 neutralizes acidic conditions of the oral cavity due to mouthwash #1 and promotes remineralization. Mouthwash #2 has sodium fluoride and is comparable to ACT®. The fluoride recruits calcium and phosphate ions to lesions in the enamel and fills the pores. After a predetermined amount of time, the user removes mouthwash #2 from the oral cavity. It is critical to maintain the order of use so that mouthwash #1 is always used before mouthwash #2 otherwise the function of the system is lost because the acidic conditions of mouthwash #1 will not be neutralized. It is also important to keep the steps separate. If the two types of washes are combined into a single solution, the user will not receive the full benefits of both rinses beyond the combined rinse. A combined rinse will have a higher pH than the acidic wash allowing bacteria to survive.

This system is improved over previous mouthwashes because it accomplishes what neither mouthwash is able to do on its own. The two step process kills bacteria, neutralizes the pH in the oral cavity and remineralizes teeth.

### DETAILED DESCRIPTION OF THE INVENTION

This invention combines the use of two separate mouthwashes in a particular sequence. The first mouthwash is acidic and kills bacteria in the oral cavity. It also loosens plaque deposits. Mouthwash #1 has the active ingredients Thymol, Eucalyptol, Menthol and Methyl Salicylate in certain amounts. Thymol, Eucalyptol and Menthol act as an antiseptic and make the solution acidic. An example of mouthwash #1 is LISTERINE®. LISTERINE® has a pH of 4.2 and includes each of the above mentioned components. It has 0.064% Thymol, 0.092% Eucalyptol and 0.042% Menthol. Another important ingredient is alcohol.

The user applies mouthwash #1 to the tooth surface by placing 15-20 ml in the oral cavity and allowing it to move over the surface of the teeth. Mouthwash #1 kills bacteria that live on the tooth surface and metabolize sugars. It also creates acidic environments in the mouth and allows demineralization to occur. Acid causes pores in the enamel to open allowing calcium and phosphate ions to leave the tooth surface. Loss of calcium and phosphate dissolves the crystal structure of hydroxyapatite and creates lesions on the tooth surface which lead to the formation of cavities. Acidic conditions also create a favorable environment for anaerobic bacteria to grow. These bacteria live in acidic environments and further create acid by metabolizing sugars in the mouth. Any bacteria not killed by mouthwash #1 are free to grow in this environment. After 30-35 seconds the user removes the acidic solution from the oral cavity.

The user applies 10-15 ml of mouthwash #2 to the tooth surface subsequent to the removal of mouthwash #1. Application should occur within 30 minutes so that saliva does not naturally raise the pH level. Mouthwash #2 is a neutral solution, with a pH of 7, that has sodium fluoride. In alternative embodiments, mouthwash #2 includes stannous fluoride or sodium monofluorophosphate. It neutralizes acidic conditions in the oral cavity caused by mouthwash #1 and prevents cavities by remineralization. ACT® mouthwash has 0.05% sodium fluoride and is similar to mouthwash #2. Those skilled in the art understand how a non-acidic solution neutralizes acidic conditions. Mouthwash #2 raises the pH in the oral cavity faster than saliva. This shortens the period of demineralization caused by mouthwash #1 and eliminates a place for bacteria to grow. Once the pH level rises to a certain point remineralization occurs.

Fluoride remineralizes teeth and hardens the surface by recruiting calcium and phosphate from saliva or the surrounding water layer to open lesions in the enamel. Free fluoride ions replace a hydroxyl group in hydroxyapatite to become fluoroapatite. Fluoroapatite is less soluble in acid than hydroxyapatite and helps the tooth to become more resistant against acid. Remineralization of the enamel helps the tooth surface to become smooth and shiny, making it difficult for bacteria to attach and grow. If any bacteria or plaque are left after application of mouthwash #1, fluoride from mouthwash #2 is incorporated into the plaque. The fluoride inactivates the bacteria so it no longer produces acid. Additionally when fluoride is concentrated in plaque it remains longer in the mouth where it stays effective.

Between applications of the two mouthwashes, the user may perform other dental hygiene steps such as brushing with toothpaste or flossing (as long as it is performed within 30 minutes after disposing of mouthwash #1). The user removes mouthwash #2 from the oral cavity after one to one and a half minutes. In the preferred embodiment the user does not eat or drink for at least 30 minutes after washing with ACT® because the sodium fluoride and its effects remain as long as it stays in contact with the tooth surface. When another solution such as water is applied subsequently, the fluoride stops working to fill pores and harden teeth. This two step process works best at night after the user brushes her teeth and before going to bed because the fluoride works the duration of sleep. Sleep dries up saliva and lowers the amount of available calcium for remineralization. However, when calcium dissolves out of the minerals during demineralization, it enters the water layer surrounding the tooth surface. Fluoride can then collect the calcium from this layer and bring it back to the lesion for remineralization. (We believe this is how demineralization works but we will not be bound by this theory.)

Although there have been no studies to support the idea that bad bacteria can be eliminated and replaced with good bacteria, it is possible that after a year of continuous treatment, one may be able to eliminate the use of mouthwash #1 and continue using mouthwash #2. The absence of bad anaerobic bacteria eliminates plaque from the surface of the tooth. Without plaque, the surface of the tooth is smooth and shiny. This type of surface is difficult for bacteria to collect on and grow.

This treatment process is intended for adolescents and older. Children below the age of 12 years old are not as susceptible to certain bacteria and do not require the use of an acidic mouthwash to kill bacteria

1. A process for treating teeth to kill bacteria and remineralize tooth crystals comprising the steps of:
applying a quantity of acidic mouthwash to the teeth for a first period of time in order to kill bacteria and open pores in tooth surface;
removing the acidic mouthwash and leaving the mouth at a residual acidic level; and
while the oral cavity of the mouth is in a residual, acidic state, applying a remineralizing mouthwash to the teeth comprising one or more promoters for enhancing the absorbing and crystallization of said tooth crystal ions into the tooth crystals, for a second period of time in order to neutralize the residual acidic level and to promote the remineralization of tooth crystals.

2. The method of claim 1 wherein the pH of the acidic mouthwash is between 3 and 5.

3. The method of claim 2 wherein the pH of the acidic mouth wash is at or about 4.2.

4. The method of claim 1 wherein the amount of acidic mouthwash is in a quantity between 15 to 20 ml.

5. The method of claim 1 wherein the acidic mouthwash remains in the oral cavity for a time between 30 and 35 seconds.

6. The method of claim 1 wherein the pH of the remineralizing mouthwash is about 7.

7. The method of claim 1 wherein the amount of remineralizing mouthwash is in a quantity between 10 and 15 ml.

8. The method of claim 1 wherein the remineralizing mouthwash remains on the teeth for a time between 1 and 1.5 minutes.

9. The method of claim 1 wherein the remineralizing mouthwash comprises fluoride.

10. The method of claim 9 wherein the fluoride remains on the teeth for a time over 30 minutes.

11. The method of claim 9 wherein the fluoride is stannous fluoride, sodium fluoride or sodium monofluorophosphate.

12. The method of claim 1 wherein the acidic mouthwash comprises one or more acids selected from the group consisting of eucalyptol, thymol, menthol, or methyl salicylate.

13. The method of claim 1 wherein the step of applying the remineralizing mouthwash to the teeth is carried out within 30 minutes of the step of applying a quantity of acidic mouthwash to the teeth.

14. A process for treating teeth comprising steps in the sequence set forth:
applying an acidic solution to the surface of teeth in the oral cavity, said acidic solution comprising Thymol, Eucalyptol and Menthol wherein solution has an antibacterial effect that kills oral bacteria and opens pores in the teeth allowing the release of calcium and phosphate ions;
removing said acidic solution from the oral cavity;
applying a non-acidic solution to the surface of teeth subsequent to said acidic solution; said non-acidic solution comprising sodium fluoride to neutralize acidic conditions in the mouth, prevent oral bacterial re-growth and remineralize teeth by filling open pores with calcium and phosphate; and
removing said non-acidic solution from the oral cavity.

15. A process for treating teeth comprising steps in the sequence set forth:
a. rinsing mouth thoroughly with 20 ml of Listerine for 30 seconds;
b. disposing of said Listerine;
c. rinsing mouth thoroughly with 10 ml of Act for one minute so that said Act comprising fluoride remineralizes teeth by filling open pores with calcium and organic phosphate and neutralizing acidic conditions in the mouth; and
d. disposing of said Act.
